(19) United States
(12) Patent Application Publication
(10) Pub. No.: US 2016/0029946 A1
(43) Pub. Date: Feb. 4, 2016

(54) WAVELET ANALYSIS IN NEURO DIAGNOSTICS

(71) Applicants: Adam J. SIMON, (US); Hashem ASHRAFIUON, Wayne, PA (US); Parham GHORBANIAN, (US)

(72) Inventors: Adam J. SIMON, Yardley, PA (US); Hashem ASHRAFIUON, Wayne, PA (US); Parham GHORBANIAN, Wayne, PA (US)

(21) Appl. No.: 14/777,030

(22) PCT Filed: Mar. 14, 2014
(86) PCT No.: PCT/US14/27480

§ 371 Date: Sep. 15, 2015

Related U.S. Application Data
(60) Provisional application No. 61/799,639, filed on Mar. 15, 2013.

(51) Int. Cl. A61B 5/00; A61B 5/04; A61B 5/0476

(52) U.S. Cl. CPC A61B 5/4088; A61B 5/0476; A61B 5/04012; A61B 5/726; A61B 5/7203

(57) ABSTRACT

A method of extracting brain frequency sub bands corresponding to a medical condition such as Alzheimer's Disease from EEG time series data of a patient includes the steps of applying wavelet transforms to the EEG time series data to generate a continuous wavelet transformation time series at each wavelet scale, calculating Wavelet Entropy (WE) and Sample Entropy (SE) directly from the Continuous Wavelet Transformation time series at each wavelet scale, calculating arithmetic or geometric means and accumulations across scale ranges of interest; and selecting data from major brain frequency sub-bands as candidate sets of extraction features for analysis as a diagnostic signature for the medical condition. Diagnostic signatures for Alzheimer's disease are found when values of WE or SE are in certain ranges when EEG data is collected and analyzed in connection with certain analytical tasks such as an Eyes Open task.

### CROSS REFERENCE TO RELATED APPLICATION

This application claims benefit of U.S. Provisional Application No. 61/799,639 filed Mar. 15, 2013. The content of that patent application is hereby incorporated by reference in its entirety.

### TECHNICAL FIELD

The invention relates to diagnosis and analysis of brain health through the use of activated tasks and stimuli in a system to dynamically assess one's brain state and function.

### BACKGROUND

Normal functioning of the brain and central nervous system is critical to a healthy, enjoyable and productive life. Disorders of the brain and central nervous system are among the most dreaded of diseases. Many neurological disorders such as stroke, Alzheimer's disease, and Parkinson's disease are insidious and progressive, becoming more common with increasing age. Others such as schizophrenia, depression, multiple sclerosis and epilepsy arise at younger age and can persist and progress throughout an individual's lifetime. Sudden catastrophic damage to the nervous system, such as brain trauma, infections and intoxications can also affect any individual of any age at any time.

Most nervous system dysfunction arises from complex interactions between an individual's genotype, environment and personal habits and thus often presents in highly personalized ways. However, despite the emerging importance of preventative health care, convenient means for objectively assessing the health of one's own nervous system have not been widely available. Therefore, new ways to monitor the health status of the brain and nervous system are needed for normal health surveillance, early diagnosis of dysfunction, tracking of disease progression and the discovery and optimization of treatments and new therapies.

Unlike cardiovascular and metabolic disorders, where personalized health monitoring biomarkers such as blood pressure, cholesterol, and blood glucose have long become household terms, no such convenient biomarkers of brain and nervous system health exist. Quantitative neurophysiological assessment approaches such as positron emission tomography (PET), functional magnetic resonance imaging (fMRI) and neuropsychiatric or cognition testing involve significant operator expertise, inpatient or clinic-based testing and significant time and expense. One potential technique that may be adapted to serve a broader role as a facile biomarker of nervous system function is a multi-modal assessment of the brain from a number of different forms of data, including electroencephalography (EEG), which measures the brain's ability to generate and transmit electrical signals. However, formal lab-based EEG approaches typically require significant operator training, cumbersome equipment, and are used primarily to test for epilepsy.

Alternate and innovative biomarker approaches are needed to provide quantitative measurements of personal brain health that could greatly improve the prevention, diagnosis and treatment of neurological and psychiatric disorders. Unique multi-modal devices and tests that lead to biomarkers of Parkinson's disease, Alzheimer's disease, concussion and other neurological and neuropsychiatric conditions is a pressing need.

### SUMMARY

The present invention relates to methods of signal processing and analysis associated with using wavelet transformations in both a discrete and continuous fashion. One particular embodiment of the present invention involves a novel approach where one calculates the Wavelet Entropy (WE) and the Sample Entropy (SE) directly from the Continuous Wavelet Transformation time series at each wavelet scale and then in a second step, one calculates the arithmetic or geometric means and accumulations across scale ranges of interest. These ranges could be advantageously chosen to corresponding to the major brain frequency sub-bands of the spectral signal processing literature.

Another embodiment of the present invention includes the calculation of the Wavelet Entropy (WE) that approximately corresponds to the standard sub-bands of the spectral signal processing literature. In one embodiment, the WE for each of the delta upper, theta, alpha, and beta sub-bands are calculated and subsequently used as a candidate set of extracted features from the time series under analysis.

Another embodiment of the present invention includes the calculation of the Sample Entropy (SE) when applied to the time series representing the wavelet coefficients at each scale after Continuous Wavelet Transformation rather than to the raw EEG voltage as a function of time.

Yet another embodiment of the present invention includes removing areas of artifact from a time series by nullifying an artifact region and then reconstructed the nulled samples using FFT interpolation of the trailing and subsequent recorded data.

Particular embodiments of the present invention include the utilization of any one of the following features for any diagnostic signature or purpose related to Alzheimer's disease: the wavelet coefficient in the D3 scale range during a binaural beat auditory stimulation at beat frequency of 18 Hz; the skewness of the D2 and/or D3 scale during the One Card Learning cognitive task (CG3), the skewness of D3 during the CogState Attention (CG1) task, or the kurtosis of the D5 scale during the PASAT task, in particular with 2.0 s interval (P2.0).

Still other embodiments of the invention include use of signatures or features that include the relative mean powers of the wavelet scales corresponding to theta_upper sub-band during CG3 (p=0.040), the Wavelet Entropy (WE) of the scales corresponding to delta_upper sub-band during AS1 (p=0.006), and the skewness of wavelet scale ranges corresponding to alpha sub-band during AS3 (p=0.034).

An important result of the invention is that the Wavelet Entropy (WE) of continuous wavelet transform (CWT) scale ranges corresponding to the alpha sub-band is significantly lower for AD compared to CTL subjects during an Eyes Open task (EO4) and/or an Eyes Closed task (EC5). In addition, the Sample Entropy (SE) of CWT scale ranges corresponding to the beta sub-band during an Eyes Closed task (EC3) and theta sub-band during an Eyes Open task (EO4, EO6) or Eyes Closed task (EC5) are significantly lower for AD regardless of the wavelet function compared to Control CTL.

In addition, an aspect of the present invention includes, in either a univariate or standalone classifier or as part of a multivariate signature, the standard deviation of CWT coefficients corresponding to theta sub-band during an Eyes Open task (EO4) when it is greater than 1.91 arb, then the subject is predicted to have AD from a decision tree.

Another embodiment of the present invention includes a decision tree or other predictive model that includes the wavelet entropy (WE) of CWT coefficients corresponding to 8-13 Hz (˜alpha sub-band) during an Eyes Open task (EO4) and, if this value is less than 1.6 arb, then the subject is predicted to have AD.

In yet another embodiment of the present invention, a decision tree or other predictive model includes the wavelet entropy (WE) of CWT coefficients corresponding to 2-4 Hz (delta_upper sub-band) during a binaural beat auditory stimulation task (AS1) and if this value for a subject is less than 2.63 arb, then the predictive model would identify this subject as an AD patient. Otherwise, if the skewness value of the wavelet coefficients corresponding to 2-4 Hz from an Eyes Open task (EO4) is less than −0.022 arb, then the subject is predicted to be an AD patient. dr

### BRIEF DESCRIPTION OF THE DRAWINGS

Embodiments of the invention can be better understood with reference to the following drawings.

FIG. 1 is a graphical presentation of the raw EEG signal of subject 11 before (top “Raw EEG”) and after (bottom “Filtered EEG”) artifact detection pre-processing. Y-axis is arbitrary units from the onboard 10 bit unsigned Analog to Digital Converter (ADC). Two enlargements from the main time series can be visualized at greater detail both before and after artifact detection.

FIG. 2 is a top down schematic diagram illustrating five level decomposition of an EEG signal where D1-D5 and AS are the DWT representation of the signal.

FIG. 3 is a graphical presentation of the EEG signal and its DWT decompositions for CTL subject 5, EO4 block

FIG. 4 is a graphical presentation of the EEG signal and its DWT decompositions for AD subject 25, EO4 block.

FIG. 5 is a graphical representation of an optimal decision tree for resting conditions, where x is the mean power of D4 of the second eyes-open state (EO4) and is also a statistically significant feature of AD patients. The values within parentheses indicate the number of properly classified subjects.

FIG. 6 is a graphical representation of an optimal decision tree result for active states. xl is the minimum value of D3 of auditory stimulation at 18 Hz (AS3), x2 is the skewness of D5 of PASAT 2.4 s interval (P2.4), and x3 is the kurtosis of D5 of PASAT 2.0 s interval (P2.0). Only x1 and x3 are statistically significant. The values within parentheses indicate the number of classified subjects.

FIG. 7 is a graphical representation of an optimal decision tree result using all recording blocks. x1 is the minimum value of D3 of auditory stimulation at 18 Hz (AS3), x2 is the mean power of D4 of the first eyes-open state (EO2), and x3 is the kurtosis of D5 of PASAT 2.0 s interval (P2.0). Only x1 and x3 are statistically significant. The values within parentheses indicate the number of classified subjects. The values within parentheses indicate the number of classified subjects.

FIG. 8A is a graphical representation of the raw EEG signal of subject 2 during EO4 before artifact detection and removal.

FIG. 8B is a graphical representation of the raw EEG signal of subject 2 during EO4 after artifact detection and removal.

FIG. 9 is a graphical representation of the top line decision tree where x is the absolute mean power of wavelet scales corresponding to theta sub-band during E04 task.

FIG. 10 is a graphical representation of the decision tree after removal of the most dominant feature where x is the standard deviation value of wavelet scales corresponding to theta sub-band during EO4 task.

FIG. 11 is a graphical representation of the decision tree after removal of the first two most dominant discriminating features where x is wavelet entropy of wavelet scales corresponding to the alpha sub-band during EC5 task.

FIG. 12 is a graphical representation of the decision tree after removal of the first three dominant discriminating features, where x1 is the wavelet entropy of the wavelet scales corresponding to delta-upper sub-band during AS1 task and x2 is the skewness of the wavelet scales corresponding to delta-upper sub-band during EO4 task.

### DETAILED DESCRIPTION OF ILLUSTRATIVE EMBODIMENTS

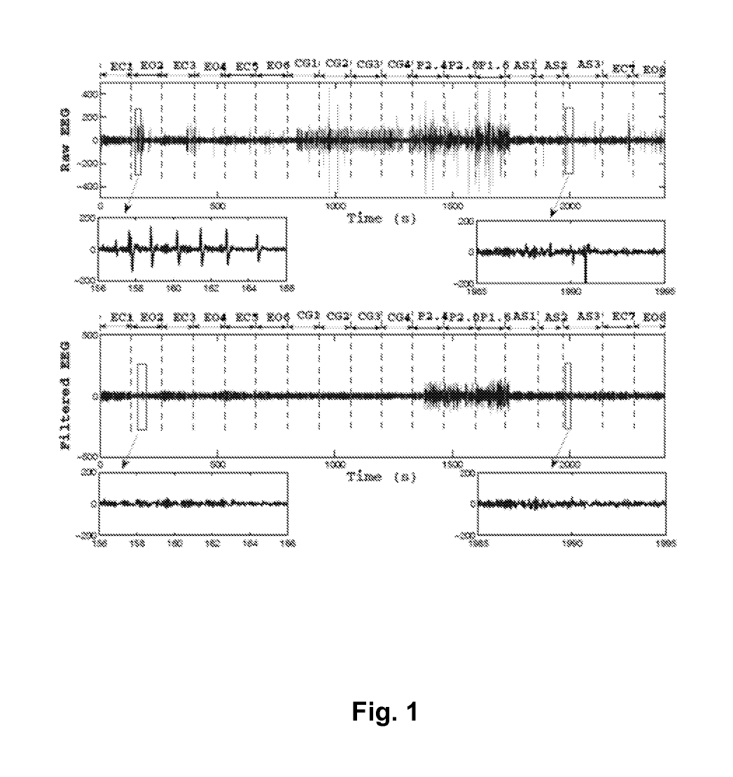

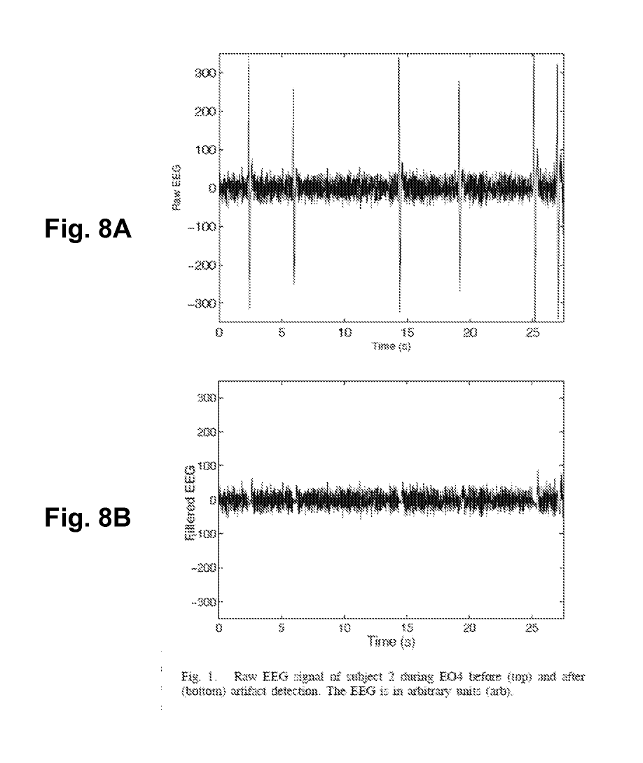

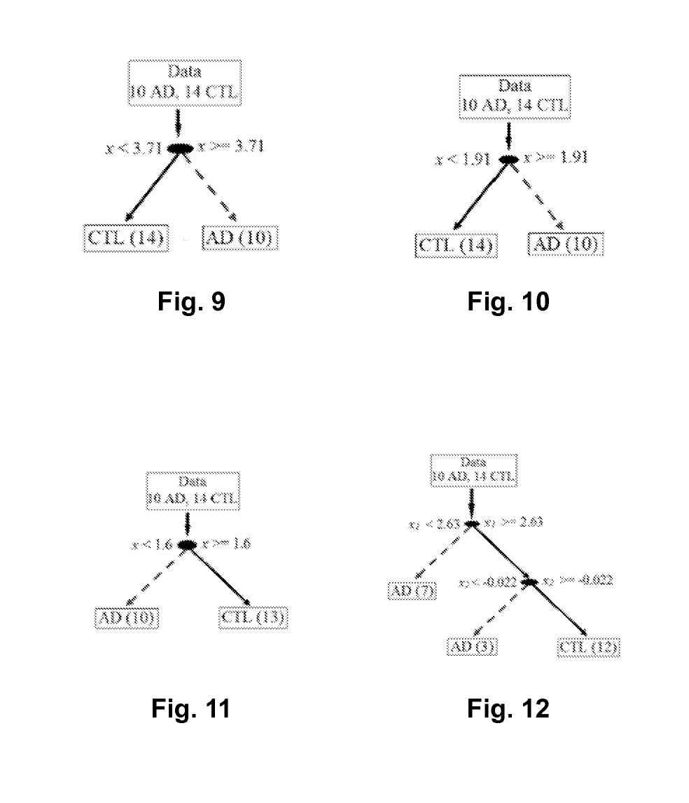

The invention will be described in detail below with reference to FIGS. 1-12. Those skilled in the art will appreciate that the description given herein with respect to those figures is for exemplary purposes only and is not intended in any way to limit the scope of the invention. All questions regarding the scope of the invention may be resolved by referring to the appended claims.

### Definitions

By “electrode to the scalp” we mean to include, without limitation, those electrodes requiring gel, dry electrode sensors, contactless sensors and any other means of measuring the electrical potential or apparent electrical induced potential by electromagnetic means.

By “monitor the brain and nervous system” we mean to include, without limitation, surveillance of normal health and aging, the early detection and monitoring of brain dysfunction, monitoring of brain injury and recovery, monitoring disease onset, progression and response to therapy, for the discovery and optimization of treatment and drug therapies, including without limitation, monitoring investigational compounds and registered pharmaceutical agents, as well as the monitoring of illegal substances and their presence or influence on an individual while driving, playing sports, or engaged in other regulated behaviors.

A “medical therapy” as used herein is intended to encompass any form of therapy with potential medical effect, including, without limitation, any pharmaceutical agent or treatment, compounds, biologics, medical device therapy, exercise, biofeedback or combinations thereof.

By “EEG data” we mean to include without limitation the raw time series, any spectral properties determined after Fourier transformation, any nonlinear properties after non-linear analysis, any wavelet properties, any summary biometric variables and any combinations thereof.

A “sensory and cognitive challenge” as used herein is intended to encompass any form of sensory stimuli (to the five senses), cognitive challenges (to the mind), and other challenges (such as a respiratory CO₂ challenge, virtual reality balance challenge, hammer to knee reflex challenge, etc.).

A “sensory and cognitive challenge state” as used herein is intended to encompass any state of the brain and nervous system during the exposure to the sensory and cognitive challenge.

An “electronic system” as used herein is intended to encompass, without limitation, hardware, software, firmware, analog circuits, DC-coupled or AC-coupled circuits, digital circuits, FPGA, ASICS, visual displays, audio transducers, temperature transducers, olfactory and odor generators, or any combination of the above.

By “spectral bands” we mean without limitation the generally accepted definitions in the standard literature conventions such that the bands of the PSD are often separated into the Delta band (f<4 Hz), the Theta band (4<f<7 Hz), the Alpha band (8<f<12 Hz), the Beta band (12<f<30 Hz), and the Gamma band (30<f<100 Hz). The exact boundaries of these bands are subject to some interpretation and are not considered hard and fast to all practitioners in the field.

By “calibrating” we mean the process of putting known inputs into the system and adjusting internal gain, offset or other adjustable parameters in order to bring the system to a quantitative state of reproducibility.

By “conducting quality control” we mean conducting assessments of the system with known input signals and verifying that the output of the system is as expected. Moreover, verifying the output to known input reference signals constitutes a form of quality control which assures that the system was in good working order either before or just after a block of data was collected on a human subject.

By “biomarker” we mean an objective measure of a biological or physiological function or process.

By “biomarker features or metrics” we mean a variable, biomarker, metric or feature which characterizes some aspect of the raw underlying time series data. These terms are equivalent for a biomarker as an objective measure and can be used interchangeably.

By “non-invasively” we mean lacking the need to penetrate the skin or tissue of a human subject.

By “diagnosis” we mean any one of the multiple intended use of a diagnostic including to classify subjects in categorical groups, to aid in the diagnosis when used with other additional information, to screen at a high level where no a priori reason exists, to be used as a prognostic marker, to be used as a disease or injury progression marker, to be used as a treatment response marker or even as a treatment monitoring endpoint.

By “electronics module” or “EM” or “reusable electronic module” or “REM” or “multi-functional biosensor” or “MFB” we mean an electronics module or device that can be used to record biological signals from the same subject or multiple subjects at different times. By the same terms, we also mean a disposable electronics module that can be used once and thrown away which may be part of the future as miniaturization becomes more common place and costs of production are reduced. The electronics module can have only one sensing function or a multitude (more than one), where the latter (more than one) is more common. All of these terms are equivalent and do not limit the scope of the invention.

By “biosignals” we mean any direct or indirect biological signal measurement data streams which either directly derives from the human subject under assessment or indirectly derives from the human subject. Non-limiting examples for illustration purposes include EEG brainwave data recorded either directly from the scalp or contactless from the scalp, core temperature, physical motion or balance derived from body worn accelerometers, gyrometers, and magnetic compasses, the acoustic sound from a microphone to capture the voice of the individual, the stream of camera images from a front facing camera, the heart rate, heart rate variability and arterial oxygen from a would pulse oximeter, the skin conductance measured along the skin, the cognitive task information recorded as keyboard strokes, mouse clicks or touch screen events. There are many other biosignals to be recorded as well.

### Wavelet Analysis Algorithms

After one conducts artifact signal pre-processing, it is often of interest to process the raw time series data through any number of commonly used techniques. For instance, in the EEG literature, it is common to utilize time series analysis (Gabor), spectral analysis (Fast Fourier Transformation) and Non-linear dynamics analysis (Lyaponouv exponents, entropy, and dimensionality). In addition, a fruitful additional avenue of signal processing includes wavelet transformations.

As a non-limiting example, an EEG signal is comprised of transient oscillations across a number of frequencies. Microphone recordings, accelerometer measurements and other biosignal data streams can be similarly analyzed. Decomposition of the EEG signal using a Fast Fourier transform (FFT) based power spectral approach continues to be a widely used analytic approach to extract features that can potentially aid with predicting AD or other disease state. General findings point to slowing of EEG in AD patients as measured by increased power in the lower frequency delta (1-4 Hz) and theta (4-8 Hz) sub-bands and decreased power in higher frequency sub-bands alpha (8-13 Hz) and beta (13-30 Hz).

Since EEG signals are non-stationary frequency based, methods such as FFT may not be effective tools for their analysis. Meanwhile, time domain nonlinear dynamics approaches are computationally complex and have not yet demonstrated reliable diagnostic power. A promising approach to EEG analysis is the use of wavelet functions to perform spectral analysis. Wavelet-based analysis has the advantage of estimating the power of transient signals without a loss of frequency resolution. Both continuous wavelet transform (CWT) and discrete wavelet transform (DWT) have been used to analyze EEG and various other signal activity. DWT is generally more computationally efficient than CWT. On the other hand, when judiciously employed, CWT can clarify subtle information that DWT cannot extract. It has also been shown that the high redundancy of the CWT approach can be used for precise localization of event-related brain potential data components in the time-frequency domain and, thus, turned into an advantage.

### Clinical Study

The objective of this study was to identify the discriminant features of EEG signals extracted from Alzheimer's disease (AD) patients compared to healthy age-matched control subjects. The study design was an initial device, single visit parallel-group, multi-center trial. Up to 250 subjects were to get stratified into several cohorts. Inclusion criteria included: 1-healthy normal's ages; 2-diagnosis of probable AD according to the NINCDS-ADRDA Alzheimer's criteria; 3- Mini-mental state examination (MMSE) score 20-27; 4-diagnosis of mild cognitive impairment (MCI) according to Peterson criteria; 5-availability of a caregiver for AD and MCI subjects. Study exclusion criteria included: 1-diagnosis of significant neurological disease other than AD; 2-history of strokes, seizures, or traumatic brain injuries; 3-Chronic pain; and 4-use of high doses of sedating or narcotic medications. Other demographic items noted were date of birth, gender, ethnicity, education, relevant medical history, current prescription and non-prescription medications, nutritional supplements, and alcohol/tobacco use.

All Personal Health Information (PHI) was retained at Palm Drive Hospital and no PHI was provided to any collaborator for HIPAA Compliance. Subjects were assigned a random/sequential subject number which was the only identifier used to analyze the demographic, independent, and subsequently dependent variables of the study. All study data were encrypted via AES-256 bit encryption at the site of data acquisition before transport to central servers whenever any information was present in the data file. The inventors also employed a multi-step process whereby all parties remained blind until the final extracted EEG features data table was produced and circulated internally to the collaborating members.

Twenty six subjects were enrolled, one withdrew due to non-study related reasons, and one did not qualify as Alzheimer's disease (AD) or control (CTL) but was diagnosed with Mild Cognitive Impairment (MCI). Data from the remaining 24 subjects were considered, including 10 AD and 14 age-matched CTL. The subject information for these 24 individuals is presented in Table 1.

TABLE 1
Subject demographics and helth information

| Subject No. | Gender | Age | Handedness | Clinical Diagnosis |
|---|---|---|---|---|
| 1 | F | 57 | R | CTL |
| 2 | F | 86 | R | CTL |
| 3 | F | 54 | R | CTL |
| 4 | F | 68 | R | CTL |
| 5 | M | 63 | L | CTL |
| 6 | F | 83 | R | AD |
| 7 | F | 83 | R | CTL |
| 8 | F | 67 | R | CTL |
| 9 | M | 82 | R | AD |
| 10 | M | 69 | R | CTL |
| 11 | M | 75 | R | CTL |
| 12 | F | 74 | R | CTL |
| 13 | F | 75 | R | CTL |
| 14 | F | 57 | R | CTL |
| 15 | M | 81 | R | CTL |
| 16 | F | 85 | R | CTL |
| 17 | M | 84 | R | AD |
| 18 | F | 75 | R | AD |
| 19 | M | 80 | R | AD |
| 20 | M | 82 | R | AD |
| 21 | M | 73 | R | AD |
| 22 | M | 86 | R | AD |
| 23 | M | 76 | R | AD |
| 24 | F | 89 | R | AD |

Behavioral Tasks Within the Battery of Assessment

Wearing the EEG headset data collecting device, subjects were asked to sit in a comfortable chair and open and close their eyes for nearly two-minute blocks, alternately recording 3 sessions of resting eyes-closed (EC) and 3 sessions of resting eyes-open (EO). They were then tasked with the four components of the CogState Research (Melbourne, Australia) brief battery: Detection, Identification, One Card Back, and One Card Learning tasks. CogState's brief battery is a computerized neuropsychological battery designed to be sensitive to the cognitive impairments that characterize mild-to-moderate Alzheimer's disease yet simple enough for patients to complete without requiring great support or assistance. The Detection task is a measure of simple reaction time and has been shown to provide a valid assessment of psychomotor function in healthy adults with schizophrenia. The Identification task is a measure of choice reaction time and has been shown to provide a valid assessment of visual attention. The One Card Learning and One Card Back cognitive tasks are valid measures of working memory.

Next, the Paced Auditory Serial-Addition Task (PASAT) task of 60 auditory addition trials was conducted at up to 3 different lag intervals of trial. PASAT is a measure of cognitive function that specifically assesses auditory information processing speed and flexibility, as well as calculation ability. Subjects are asked to listen to a series of numbers and are requested to add consecutive pairs of numbers as they listen. There is no visual component to this task.

Brief auditory binaural beat stimulations (90 seconds, 50-75 db) with differential beat frequencies of 6 Hz, 12 Hz, and 18 Hz were conducted next, followed by one final block of each resting EC and EO to close the data collection paradigm. There was normally a short break between recording sessions. Although there were a total of 18 possible recording tasks, a large number of subjects did not complete the PASAT 1.6 (s) interval (Task 13) and hence the data from this task was not included in the analysis.

### EEG Signal Quality and Pre-processing

The rechargeable battery powered Bluetooth enabled EEG headset eliminated frequently observed artifacts including line noise. However, it was critical to detect and eliminate other artifacts such as eye-blinks in the EEG signal. These artifacts, frequent at Fp1 location, often have high amplitudes relative to brain signals. Thus, even if their appearance in the EEG data is not frequent, they may bias the results of a given block of data or experiment. In this study, any DC offset of the EEG signal was subtracted and an artifact detection pre-processing algorithm was used to eliminate large amplitude artifacts greater than 4.5 standard deviations sigma. An algorithm was developed to detect such artifacts, nullify, and then reconstruct the nulled samples using FFT interpolation of the trailing and subsequent recorded data. However, amplitude-based artifact detection method sometimes fails to detect low frequency artifacts such as small eye blinks. Hence, the inventors recursively applied the artifact detection method to the modified signal up to three times. This method eliminated the remaining low frequency artifacts with very high reliability considering that the EEG signals are generally normally distributed (i.e., 1 in 49053 samples are expected to be out of range for the filtered signal while the sample size is in the 10000 to 20000 range). For illustrative purposes, FIG. 1 shows all the recorded EEG blocks concatenated one after the other for subject number 11, a CTL subject, in arbitrary units from the 10-bit analog-to-digital converter (ADC) before and after artifact detection. The enlarged area on the left is part of the second recording state EO2 where all eye blinks have been eliminated. The enlarged area on the right shows part of the 18 Hz auditory stimulation, AS3, where a few eye blinks plus a single artifact with large amplitude has been removed. The results show improvement over previous artifact detection. However, large amplitude signals in the PASAT recordings have not been filtered out due to larger during these sessions which are due normal physiological activities since subjects respond vocally.

The headset sample rate was specified at fs=128 Hz by the manufacturer. However, the effective sample rate was closer to fs=125 Hz in the experiments. Frequencies below 1 Hz and above 60 Hz (near Nyquist frequency) were filtered out. Furthermore, the inventors only analyzed frequencies between 2 Hz and 30 Hz due to the demonstrated reliability of the device.

### Discrete Wavelet Feature Extraction Algorithms

A discrete wavelet transform was used to analyze the collected EEG signal at different temporal resolutions through its decomposition into several successive frequency bands by utilizing a scaling and a wavelet function associated with low-pass and high-pass filters. The original EEG signal x(t) forms the discrete time signal x[i], which is first passed through a half-band high-pass filter, g[i], and a low-pass filter, h[i]. Filtering followed by sub-sampling constitutes one level of decomposition and can be expressed as follows: d1[k]=Summation over n of x[i].g[2k-i] Eq. (A) and a1[k]=Summation over n of x[i].h[2k-i] eq. (B) where d₁[k] and a₁ [k] are level 1 detail and approximation coefficients at translation k, which are the outputs of the high-pass and low-pass filters after the sub-sampling, respectively. This procedure, called sub-band coding, is repeated for further decomposition as many times as desired or until no more sub-sampling is possible. At each level, it results in half the time resolution (due to sub-sampling) and double the frequency resolution (due to filtering), allowing the signal to be analyzed at different frequency ranges with different resolutions.

Of the many families of mother wavelets, the Daubechies family possesses a number of characteristics that are ideal for EEG analysis, including 1) the well understood and smoothing characteristics of Daubechies2 (db2) and 2) detection of changes in EEG important for detecting epileptiform activity. In the approach used by the inventors, five different mother wavelets from the Daubechies family were used: db2, db4, db6, db8, and db10.

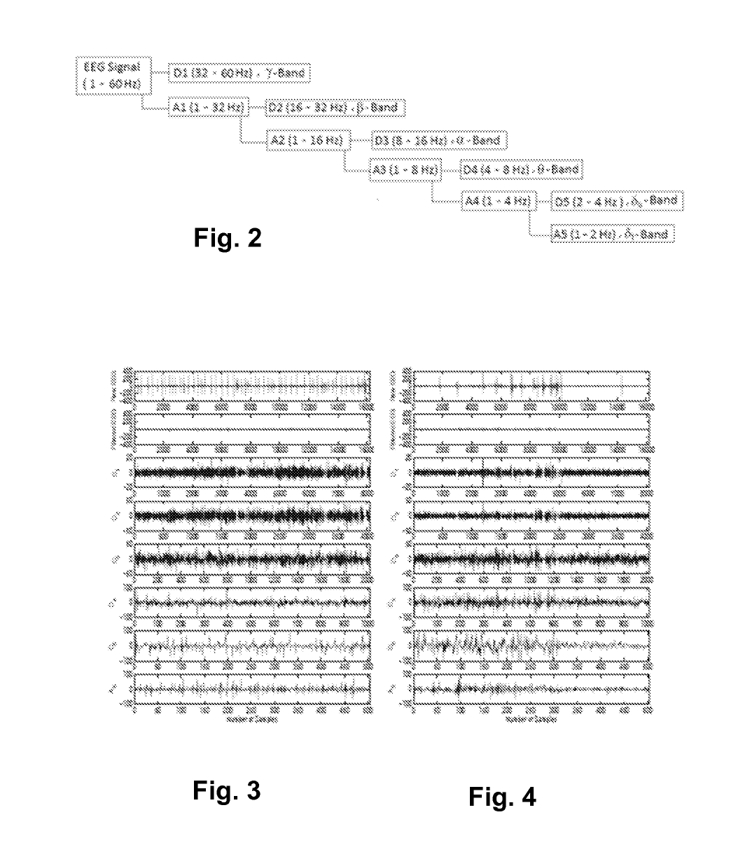

The inventors performed five levels of decomposition resulting in D1 (approximately related to the gamma spectral frequency sub-band) through D5 (approximately related to the upper delta spectral frequency sub-band) and A1 through A5 (approximately related to lower delta spectral frequency sub-band), as shown in FIG. 2. Table 2 shows the exact sub-band frequency ranges and their corresponding approximate EEG major spectral frequency bands. However, not all these sub-bands are useful and reliable. Since the recording device was validated for 2-30 Hz frequency range, the inventors excluded D1 (˜gamma) and A5 (˜lower delta) sub-band features. As a result, the effective sub-bands used in this study were D2 - D5.

TABLE 3
DWT sub-band frequencies and the corresponding
approximate major brain frequency sub-bands.

| | Frequency Range | Corresponding EEG | |
|---|---|---|---|
| Sub band | (Hz) | frequency band (Hz) | |
| D₁ | 30-60 | γ | (>30) |
| D₂ | 15-30 | β | (13-30) |
| D₃ | 7.5-15 | α | (8-13) |
| D₄ | 3.75-7.5 | θ | (4-8) |
| D₅ | 1.875-3.75 | δₕ | (2-4) |
| A₅ | 1-1.875 | δₗ | (0-2) |

Having created the DWT sub-bands of EEG signal, the inventors can extract the common statistical features from the DWT analysis. In this study, the inventors selected the minimum, maximum, mean power, as well as standard deviation (STD), skewness, and kurtosis values of the wavelet coefficients as candidate extracted features. The mean power of the wavelet coefficients was computed as follows: Pⱼ=(1/n)* Summation over i from i=0 to i=n−1 of |xi|², for j=1, . . . ,N Eq. (C) where x,'s are the computed coefficients of the signal at each sub-band, n is the number of computed coefficients at each sub-band, and N is the total number of sub-bands. These values were computed at each level of DWT decomposition separately for each recording block from each task of each subject. Note that the inventors did not consider the mean values since the mean was subtracted before processing the data.

### Results of DWT

The univariate results of the features from AD vs CTL are show in Table 3. In Table 4, one can see the number of significant features based on the choice of the mother wavelet family chosen, from db2 thru db10.

TABLE 4
Statistically significant DWT EEG features of AD subjects based on Wilcoxon
rank-sum test and their corresponding false postitive rate p-value.

| | EO1 | EO2 | EO3 | EO4 | EO5 | EO6 | GG1 | GG2 | GG3 | GG4 | P2.1 | P20 | AB1 | AB2 | AB3 | [symbol] | [symbol] |
|---|---|---|---|---|---|---|---|---|---|---|---|---|---|---|---|---|---|
| MP D₂ | — | — | [symbol] | — | [symbol] | — | — | — | — | — | — | — | — | — | — | — | — |
| MP D₃ | — | — | — | — | — | — | — | — | — | — | — | — | — | — | [symbol] | — | — |
| MP D₄ | — | — | — | [symbol] | — | — | — | — | — | — | — | — | — | — | — | — | — |
| MP D₅ | — | — | — | [symbol] | — | [symbol] | — | — | — | — | — | — | — | — | — | — | — |
| Min D₂ | — | — | — | — | — | — | — | — | — | — | — | — | — | — | — | — | — |
| Min D₃ | — | — | — | — | — | — | — | — | — | — | — | — | — | — | [symbol] | — | — |
| Min D₄ | — | — | — | [symbol] | — | — | — | — | — | — | — | — | — | — | — | — | — |
| Min D₅ | — | — | — | [symbol] | — | — | — | — | — | — | — | — | — | — | — | — | — |
| Max D₂ | — | — | — | — | — | — | — | — | — | — | — | — | — | — | — | — | — |
| Max D₃ | — | — | — | — | — | — | — | — | — | — | — | — | — | — | [symbol] | — | — |
| Max D₄ | — | — | — | [symbol] | — | — | — | — | — | — | — | — | — | — | — | — | — |
| Max D₅ | — | — | — | [symbol] | — | — | — | — | — | — | — | — | — | — | — | — | — |
| Std D₂ | — | — | [symbol] | — | — | [symbol] | — | — | — | — | — | — | — | — | — | — | — |
| Std D₃ | — | — | — | — | — | — | — | — | — | — | — | — | — | — | [symbol] | — | — |
| Std D₄ | — | — | — | [symbol] | — | — | — | — | — | — | — | — | — | — | — | — | — |
| Std D₅ | — | — | — | [symbol] | — | [symbol] | — | — | — | — | — | — | — | — | — | — | — |
| Skew D₂ | — | — | — | — | — | — | — | — | [symbol] | — | — | — | — | — | — | — | — |
| Skew D₃ | — | — | — | — | — | — | [symbol] | — | [symbol] | — | — | — | — | — | — | — | — |
| Skew D₄ | — | — | — | — | — | — | — | — | — | — | — | — | — | — | — | — | — |
| Skew D₅ | — | — | — | — | — | — | — | — | — | — | — | — | — | — | — | — | — |
| [symbol] D₂ | — | — | [symbol] | — | [symbol] | [symbol] | — | — | — | — | — | — | — | — | — | [symbol] | [symbol] |
| [symbol] D₃ | — | — | — | [symbol] | — | — | — | — | — | — | — | — | — | — | — | — | — |
| [symbol] D₄ | — | — | — | — | — | — | — | — | — | — | — | — | — | — | — | — | — |
| [symbol] D₅ | — | — | — | — | — | — | — | — | — | — | — | [symbol] | — | — | — | — | — |

[symbol]

indicates data missing or illegible when filed

TABLE 5
Number of features derived by different
Daubechies family of wavelets.

| Mother Wavelet | # of Significant features |
|---|---|
| Daubechies2 (db2) | 10 |
| Daubechies4 (db4) | 28 |
| Daubechies6 (db6) | 21 |
| Daubechies8 (db8) | 26 |
| Daubechies10 (db10) | 25 |

### Continuous Wavelet Feature Extraction Algorithms

Recently, CWT has been used in the art to extract a number of features from EEG signals in a variety of subjects. CWT was used to extract geometric mean power at different scale ranges, which are related to different major brain frequency bands. The extracted features are then used for classification of EEG signals. Various predictive statistical methods such as neural network, fuzzy systems, and support vector machine were employed in these studies. However, to the inventors' knowledge, very few studies have used CWT to extract discriminating AD features of EEG signals. Ueda et al. used the Gabor wavelet for diagnosing Alzheimer's disease (AD) and mild cognitive impairment (MCI) and reported that the variance of the power were low for AD patients in the alpha sub-band and high for MCI patients in the theta sub-band. A consideration with this approach and the wavelet transform in general is that it requires an a priori choice of a mother wavelet and estimates of spectral power depends on its scaling and shifting properties.

Nonlinear dynamic measures such as entropy have also been extensively used to analyze the EEG signal and to determine discriminants of AD. General findings from these computationally intensive studies point to lower complexity of the EEG signal in AD patients. Entropy is a thermodynamic quantity addressing randomness and predictability where greater entropy is often associated with more randomness and chaotic behavior. Biological signals often contain both deterministic and random components, so entropy has clear advantages in analyzing biological systems. The inventors use two classes of entropy, namely wavelet entropy as a measure of the flatness of frequency spectrum and sample entropy as a measure of system complexity. Wavelet Entropy and Sample Entropy of the EEG signals from control subject have been shown to be higher for control subjects than AD patients at several electrodes locations. However, only a few sample entropy features were statistically significant. As will be explained in more detail below, the inventors have developed a novel approach where Wavelet Entropy (WE) and Sample Entropy (SE) are calculated from the time series at each wavelet scale and then in a second step, their arithmetic means are calculated across scale ranges corresponding to the major brain frequency sub-bands.

The wavelet transform is an excellent method for (non-stationary) signal analysis since it represents the signal in terms of both time and frequency. For computational purposes, the CWT of a time series x(t) at discrete time location i and scale sj is defined as: where Ci,j=C(Tau_j, s_j)=1/sqrt(s_j) * integral from−infinity to +infinity of x(t) * psi star (t−Tau_i/s_j)dt (1)(Eq. 1) where Ci,j represents the wavelet coefficient at time sample i and scale s_j 6=0, x(t) is the biosignal during each recording, (t) is the wavelet function called the “mother wavelet”, and superscript “*” or “star” denotes the complex conjugate of the function according to well published methods.

### A. Choice of Mother Wavelet

There are a number of wavelet functions, the choice of which depends on the type of features to be extracted from the signal. The Morlet wavelet is the most frequently used in practice because of its simple numerical implementation and better accuracy compared to most other wavelet functions in analyzing signals such as EEG. However, the Daubechies wavelets have a number of characteristics that are in particular ideal for EEG analysis including detection of changes in EEG important for identifying epileptiform activity. Choice of mother wavelet function is the most important factor for a reliable wavelet transform analysis. Therefore, the inventors have used five mother wavelets from the Daubechies (db4, db6, db8, and db10) and Morlet wavelet functions without prejudice and let a new classifier choose the best one.

### B. Wavelet Scales and Brain Frequency Bands

The relationship between CWT scales and frequency is not precise but has a roughly inverse form such that low scale corresponds to high frequency and vice versa. However, an approximate relationship is required in order to relate the scales to the major brain spectral frequency sub-bands. In this application, the inventors use a mapping between scales and pseudo frequencies suggested by Darvishi and Al-Ani: Fj=Fc/(sj*delta) (2) where delta is the sampling period (=1/fs) (Eq. 2), Fc is the center frequency of the selected wavelet function, and Fj is the pseudo-frequency corresponding to the scale sj. The inventors have defined the major brain frequency sub-bands, delta upper, theta, alpha, and beta and their upper and lower ranges according to the pseudo frequency defined in Eq. (2), as listed in Table 5. These sub-bands were selected based on the demonstrated reliability of the recording device in the 2-30 Hz range.

TABLE II
WAVELET SCALES AND THEIR CORRESPONDING
PSEUDO-FREQUENCY AND MAJOR BRAIN
FREQUENCY SUB-BANDS.

| Scale | Scale | Pseudo-Frequency | Brain EEG |
|---|---|---|---|
| counter j | values | Range (Hz) | Sub-band |
| [21-36] | [3.5-5] | [20-30] | β_{U} |
| [36-71] | [5-8.5] | [13-20] | β_{L} |
| [21-71] | [3.5-8.5] | [13-30] | β |
| [71-86] | [8.5-10] | [10-13] | α_{U} |
| [86-116] | [10-13] | [8-10] | α_{L} |
| [71-116] | [8.5-13] | [8-13] | α |
| [116-166] | [13-18] | [6-8] | θ_{U} |
| [166-246] | [18-26] | [4-6] | θ_{L} |
| [116-246] | [13-26] | [4-8] | θ |
| [246-386] | [26-40] | [2-4] | δ_{U} |

### C. Wavelet Distribution Features

The first features defined from CWT of the EEG signals were the measures that characterize the power spectrum distributions for major brain EEG frequency sub-bands based on their corresponding scale ranges. The inventors calculated Ci,j using Eq. (1) in the range of [3.5-40] with a scale step of 0.1 for each EEG recording block of the subjects using the five selected wavelet functions. Hence, referring to Table 5, index 21 and 386 corresponds to scales 3.5 and 40, respectively. The wavelet coefficients at each scale sj are averaged over time to define the power Pj [29]: Pj=1 n n Xi=1|Ci,j|2, j=21, . . . , 386, (Eq. (3)) where n is the total number of samples times. The inventors define the first two sets of CWT features as the absolute and relative powers at each of the ten frequency ranges presented in Table 5. The absolute power of a frequency range is defined as the geometric mean of the Pj values in the corresponding scale range. The relative powers are the absolute powers normalized based on the total power within a given scale range. The inventors also calculated the standard deviation and skewness of the wavelet coefficients at each scale similar to Eq. 3 and defined their geometric means within the scale ranges corresponding to delta-upper, theta, alpha, and beta as the third and fourth set of features.

D. Novel Wavelet Entropy Features of the Present Invention

Wavelet entropy (WE), as a measure of EEG complexity, is calculated similar to the method presented by Xu et al. The wavelet entropy is given in terms of the relative wavelet energy defined as the ratio of the power at each scale and total power, WE=−Summation over j of e_j . loge_j, where e_j=P_j/Summation over j of P_j (Eq. (4)). Normally, WE is defined for the full spectrum. However, in the present invention the inventors introduce and calculate WE approximately corresponding to delta_upper, theta, alpha, beta sub-bands and use them as the fifth set of features. Thus, the summation range in Eq. 4 is over the scale counters corresponding to each selected sub-band. Note that, such categorization allows the inventors to focus on the complexity of the EEG or bio signal in different spectrums.

E. Novel Sample Entropy Features of the Present Invention

Sample Entropy (SE) is the negative natural logarithm of the conditional probability that two sequences of a time series, similar form points, remain similar at the next point. SE has already been used as a potential measure of complexity of EEG signals. However, unlike other studies, in the present invention the inventors apply SE to the time series representing the wavelet coefficients at each scale rather than the EEG signal. The inventors estimate SE corresponding to each scale j, [C_{—}1,j ,C_{—}2,j , . . . , C_n,j ], by the statistic: SE(m, r, n)=−ln (U^{m+1}(r)/U^{m}(r)) (Eq. (5)), where m is the run length, r is the tolerance window size, and U^{m}(r)=1/(n−m)(n−m−1) times Summation from I=1 to n-m of U_i. (Eq. (6)). In the above equation, U_i indicates the number of k's (1≦k≦n−m) such that the Euclidean distance between uₘ(i) and uₘ (k), k not equal i, is less than or equal r and uₘ(i)=[C_{i,j}, C_{i+1,j}, . . . , C_{i+m-1,j}].

Since the analysis resolution was not high enough to accurately distinguish the statistical differences of SE at each scale, the inventors calculated the geometric means of the SE for scale ranges corresponding to delta_upper, theta, alpha, and beta sub-bands presented in Table 5 as the sixth set of features.

Calculation of SE is highly dependent on the selection of m and r. If m is too large or r is too small, then the number of matches will be too small for confident estimation of the conditional probability. On the other hand, if m is too small and r is too large, then the number of matches will be too large and little discrimination will be detected. In this case, the inventors used r=0.25 sigma which is within the recommended range of 0.2 sigma to 0.25 sigma. However, there have been no clear range of values suggested form. Hence, the inventors experimented with values ranging from 2 to 20 and selected m=8 points due to the fact that it produced more consistent and thus reliable statistical features among different wavelet functions.

The statistical results from the CWT analysis are shown in Table 6. One can see the features with statistical significance by task by the FPR p-values shown within the table.

Comparison with Other & Traditional Methods

The inventors determined EEG features using the traditional Fast Fourier Transform (FFT) and Discrete wavelet transform (DWT). In the case of FFT, the inventors used 8 s (˜1000 sample) sliding Blackman windows and determined the absolute and relative and mean powers, standard deviation, and skewness for the frequency ranges listed in Table 5.

The inventors performed five levels of decomposition for DWT using five mother wavelets from the Daubechies family db2, db4, db6, db8, and db10, which resulted in six sub-bands. The filtered signals in four of these sub-bands approximately represented the EEG major spectral frequency bands, delta-upper, theta, alpha and beta. The inventors then extracted the mean power, standard deviation, and skewness of the wavelet coefficients as the features.

A shorter list of the common statistical features between the three approaches, the absolute mean powers of major brain sub-bands, are listed in Table 7 where only db6 wavelet results are shown for CWT and DWT approaches. It is clear that FFT is not able to capture most of the statistically significant features identified by CWT and DWT. In fact, after multiple comparison adjustments by FDR, no reliable discriminant feature could be reported. This clearly indicated that wavelet transform has a superior performance in classification of AD patients when compared to FFT.

DWT features are, however, comparable with CWT where both determine similar discriminating features. While DWT seems to identify absolute mean power as a discriminating feature, the result could not be confirmed by FDR. Another disadvantage of DWT is that it could not be used to determine features corresponding to more detailed upper and lower sub-bands.

TABLE III
STATISTICALLY SIGNIFICANT DB6 CWT EEG
FEATURES OF AD AND THEIR p-VALUE. THE
BOLD DATA INDICATE STATISTICALLY SIGNIFICANT
FEATURES AFTER FDR ADJUSTMENT.

| | EO2 | EC3 | EO4 | ECS | EO6 | AS1 |
|---|---|---|---|---|---|---|
| rel δ₀ | — | — | .030 | — | — | — |
| rel θ₁ | — | — | 8−e5 | .034 | — | — |
| rel θᵤ | — | — | — | — | — | — |
| rel 0 | .026 | — | .0003 | .002 | .008 | — |
| rel α₁ | — | — | — | — | — | — |
| rel αᵤ | — | — | .004 | — | — | — |
| rel α | — | — | .019 | — | — | — |
| rel β₁ | — | — | .001 | .029 | .022 | — |
| rel βᵤ | .046 | — | .0015 | — | .009 | — |
| rel β | — | — | .001 | .029 | .022 | — |
| abs δᵤ | — | — | .001 | — | .04 | — |
| abs θ₁ | — | — | .0001 | — | .009 | — |
| abs θᵤ | .026 | .046 | .0002 | .007 | — | — |
| abs θ | — | — | 4e−5 | .015 | .013 | — |
| abs α₁ | — | — | — | — | — | — |
| abs αᵤ | — | — | — | — | — | — |
| abs α | — | — | — | — | — | — |
| abs β₁ | — | — | .005 | .013 | — | — |
| abs βᵤ | .035 | — | .004 | — | .022 | — |
| abs β | — | — | .004 | .013 | .03 | — |
| std δᵤ | — | — | .0008 | — | .04 | — |
| std θ | — | — | 4e−5 | .018 | .009 | — |
| std α | — | — | — | — | — | — |
| std β | — | — | .004 | .015 | .03 | — |
| skew.δᵤ | — | — | .03 | — | — | — |
| skew.θ | — | — | — | — | — | — |
| skew.α | — | — | — | — | — | — |
| skew.β | — | — | — | — | — | — |
| WE δᵤ | — | — | — | — | — | .006 |
| WE θ | — | — | — | — | — | — |
| WE α | — | — | .001 | .0006 | .019 | — |
| WE β | — | — | .022 | — | — | — |
| SE δᵤ | — | — | .026 | — | — | — |
| SE θ | — | — | .026 | .047 | .035 | — |
| SE α | — | — | — | — | — | — |
| SE β | — | .016 | — | — | — | — |

TABLE IV
STATISTICALLY SIGNIFICANT EEG FEATURES OF
AD AND THEIR p-VALUE FOR DIFFERENT METHODS.

| Method | Feature | EO2 | EC3 | EO4 | EC5 | EO6 |
|---|---|---|---|---|---|---|
| FFT | abs. δᵤ | — | — | — | — | — |
| | abs. θ | — | — | .04 | .009 | — |
| | abs. α | — | — | — | — | — |
| | abs. β | — | — | — | — | — |
| CWT (db6) | abs. δᵤ | — | — | .001 | — | .04 |
| | abs. θ | — | — | 4e−5 | .015 | .013 |
| | abs. α | — | — | — | — | — |
| | abs. β | — | — | .004 | .013 | .03 |
| DWT (db6) | abs. δᵤ | — | — | .0005 | — | .04 |
| | abs. θ | .03 | — | 1e−5 | .001 | — |
| | abs. α | — | — | .022 | .04 | — |
| | abs. β | .04 | — | .003 | — | .03 |

TABLE V
INDEX VALUES AFTER REMOVAL OF THE
FIRST TWO DOMINANT FEATURES.

| — | Gini | Twoing | Deviance |
|---|---|---|---|
| Morlet | 8 | 8 | 8 |
| Db4 | 2 | 2 | 2 |
| Db6 | 2 | 2 | 2 |
| Db8 | 2 | 2 | 2 |
| Db10 | 12 | 8 | 12 |

TABLE VI
INDEX VALUES AFTER REMOVAL OF THE
FIRST THREE DOMINANT FEATURES.

| — | Gini | Twoing | Deviance |
|---|---|---|---|
| Morlet | 8 | 8 | 8 |
| Db4 | 6 | 10 | 6 |
| Db6 | 5 | 5 | 5 |
| Db8 | 14 | 14 | 14 |
| Db10 | 14 | 14 | 14 |

### EXAMPLES

While the above description contains many specifics, these specifics should not be construed as limitations on the scope of the invention, but merely as exemplifications of the disclosed embodiments. Those skilled in the art will envision many other possible variations that are within the scope of the invention. The following examples will be helpful to enable one skilled in the art to make, use, and practice the invention.

### Example 1: Clinical Study and Data Collection

A. EEG Headset Characterization and Validation

A novel EEG headset device was modified for use in a clinical context to record a 128 samples/sec 10-bit data stream transmitted from the single EEG sensor placed at position Fp1 (based on a 10-20 electrode placement system). Differential voltage signals relative to the mastoid on the left ear were amplified via an application-specific integrated circuit (ASIC) containing an instrumentation differential amplifier followed by an analog filter with common mode rejection at 60 Hz. Two mastoid electrodes (reference and ground) were embedded in the left ear cup of the headset for compression contact to the left ear of the subject. After analog to digital conversion with a 10-bit unsigned analog-to-digital-converter (ADC), digital EEG signals passed through a digital signal processor before being transmitted via Bluetooth to a nearby computer.

Analytical bench studies verified the device achieving good signal-to-noise ratio. To compare the headset to traditional clinical EEG equipment, the inventors simultaneously recorded arbitrary waveform signals loaded into the buffer of a function generator hardwired in parallel to a Compumedics Neuroscan NuAmps system and the headset device. Publicly available reference EEG traces were uploaded into the Arb buffer and spooled out. After independent analysis of the recorded 10,000 samples/sec, 24-bit ADC signal from the Fp1 channel of the NuAmps system and the 128 samples/sec, 10-bit ADC output from the headset device, the gross spectral response was indistinguishable except for frequencies below 2 Hz. The analytical bench assessment demonstrated excellent ability to accurately record EEG signals in the 1-100 nV and 2-30 Hz ranges. The headset sample rate was specified at fs=128 Hz. However, the effective sample rate was closer to fs=125 Hz in the experiments. Frequencies below 1 Hz and above 60 Hz (near Nyquist frequency) were filtered out. However, the inventors only analyzed frequencies in the 2-30 Hz due to the headset device's demonstrated reliability.

The inventors investigated the integrity of EEG recordings by the device from human subjects. As the active electrode sits at position Fp1 just above the left eye on the forehead and mastoid, it was referenced via three surface contact electrodes on the left ear. The inventors recorded EEG signals sequentially from the same subject in both the resting eyes-closed (EC) and eyes-open (EO) conditions and computed the EC/EO ratio between the two power spectra. As expected, a statistically significant prominent peak of rhythm activity was observed centered around 10 Hz in the EC condition.

B. Behavioral Tasks and Clinical Study

The objective of this study was to identify the discriminant features of EEG signals extracted from Alzheimer's disease (AD) patients compared to healthy age-matched control subjects. Up to 250 subjects were to get stratified into several cohorts. Inclusion criteria included: 1-healthy normal's ages; 2—diagnosis of probable AD; 3—Mini-mental state examination; 4—diagnosis of MCI; 5—availability of a caregiver for AD and MCI subjects. Study exclusion criteria included: 1—diagnosis of significant neurological disease other than AD; 2—history of strokes, seizures, or traumatic brain injuries; 3—Chronic pain; and 4—use of high doses of sedating or narcotic medications. Other demographic items noted were date of birth, sex, ethnicity, education, relevant medical history, current prescription and non-prescription medications, nutritional supplements, and alcohol/tobacco use. All Personal Health Information (PHI) was retained at Palm Drive Hospital and no PHI was provided to any collaborator for HIPAA Compliance.

Twenty six subjects were selected, one withdrew and one did not qualify as Alzheimer's disease (AD) or control (CTL). Data from the remaining N=24 subjects were considered, including 10 AD and 14 age-matched CTL. There were 13 female and 11 male subjects with ages ranging from 57 to 89 years old. Wearing the device, subjects were asked to open and close their eyes for typically 90-second blocks, alternately recording 6 sessions under resting EC and EO conditions. They were then tasked with four components of the CogState Research (Melbourne, Australia) brief battery: Detection, Identification, One Card Back, and One Card Learning tasks. Next, the Paced Auditory Serial-Addition Task (PASAT) task of 60 auditory addition trials was conducted at up to 3 different lag intervals of trial. Brief auditory binaural beat stimulations (90 seconds, 50-75 db) with beat frequencies of 6 Hz, 12 Hz, and 18 Hz were conducted next, followed by two more blocks of resting EC and EO to close the data collection paradigm. Although there were a total of 18 possible recording tasks, a large number of subjects did not complete the PASAT 1.6 (s) interval (Task 13) and hence the data from this task was not included in the analysis.

C. EEG Signal Quality and Pre-processing

The EEG device eliminated frequently observed artifacts including line noise. However, a novel artifact detection pre-processing algorithm was developed to eliminate eye blinks and other large amplitude artifacts greater than 4.5 sigma (standard deviation). The algorithm nullified and reconstructed the nulled samples using FFT interpolation of the trailing and subsequent recorded data. For illustrative purposes, FIG. 1 shows the recorded EEG block during EO4 for subject number 2, a CTL subject, in arbitrary units from the 10-bit analog-to-digital converter (ADC) before and after artifact detection where all artifacts (mainly eye blinks) have been eliminated.

The inventors calculate EEG features using five mother wavelets in order to overcome this a priori choice of mother wavelet consideration. In this study, the inventors applied five different CWT to EEG recordings from 10 AD patients and 14 healthy age matched CTL subjects during 17 different resting and active brain conditions. The inventors computed the absolute and relative geometric mean powers, standard deviations, skewness, wavelet entropy, and sample entropy of wavelet coefficients at scale ranges corresponding to the major brain frequency sub-bands, as features. A large number of discriminating features of AD patients were identified using the applied the nonparametric Wilcoxon rank-sum statistical testing method to a large number features and corrected for multiple comparisons through False Discovery Rate control test. Multivariate analysis of variance (ANOVA) was also applied to determine the degree of correlation between the features. Decision tree algorithms were then employed to classify the most significant EEG features of AD patients. Finally, the inventors developed a new index to choose most accurate discriminating EEG features of AD patients among those classified by different decision tree algorithms for the variety of utilized wavelet transforms based on statistical significance of the features and rate of false classification.

### Example 2: Significant Discrete Wavelet Transformation Features

Choice of mother wavelet function is the most important factor for a reliable DWT analysis. Therefore, the inventors determined EEG features of AD patients compared to CTL subjects across five wavelet functions from the Daubechies family. The number of statistically significant EEG features of AD patients compared to CTL subjects, identified by the five different wavelets, are shown in Table 4, where many features were common among the different wavelet functions. The inventors then performed univariate and multivariate ANOVA for all features, applied three different split criteria, and chose the best decision tree based on reliability of the utilized features.

A. Univariate Statistics

Common statistical methods that rely on normal distribution were not applicable in the study. Therefore, the inventors used the nonparametric Wilcoxon rank-sum test, which is the two sample version of the Kruskal-Wallis one-way analysis of variance (ANOVA) by ranks. The null hypothesis of the method is that the populations from which the samples originate have the same median. The test does not identify how many differences actually occur or where they occur.

Initially, the inventors performed univariate analysis (false positive rate p<0.05) on the six sets of features extracted from the seventeen recording sessions based on each of the five different wavelet functions. Since, in each case, a large number of pairwise statistical tests (612) have been performed, multiple comparison adjustment may be applied to reduce the possibility of spurious significant results. Hence, the inventors applied False Discovery Rate (FDR) for multiple comparisons for more rigorous verification of the statistical significant features. Note that, these multiple comparison corrections are not strictly required in exploratory analysis and do not prove the significance of the findings. Nonetheless, they minimize the likelihood of the occurrence of false significant findings.

The inventors initially applied univariate statistical testing to identify the statistically significant discriminant DWT extracted features of AD patients compared to CTL subjects. Given that data within the 6 statistical measures (minimum, maximum, STD, skewness, kurtosis, and mean power) were not normally distributed, the non-parametric Wilcoxon rank-sum test for one-way ANOVA was used. Table 3 provides an overview of the db4-based DWT coefficient features extracted during these tasks that are statistically different with their corresponding false positive rate p-values. Overall, the second eyes-open state (EO4) yielded the most number of statistically significant features followed by the third eyes-open state (EO6) and auditory stimulation at 18 Hz (AS3). Note that, the differences in the first and last round of resting states can be explained by the fact that the subjects may not have initially been fully resting and were tired and restless at the end of recording sessions. The other four resting states combine to yield similar results to their individual recording blocks.

All statistically significant features of AD patients observed in the resting EO and EC are consistent with published literature where increased delta and theta activities and decreased beta activity have been reported for AD patients. To illustrate the performance of DWT with db4 wavelet function, FIGS. 3 and 4 show the raw EEG signal recorded during EO4 followed by the signals after each level of decomposition for subjects 5 (a CTL subject) and 25 (an AD subject), respectively. The higher D5 (˜delta) and D4 (˜theta) activities and lower D3 (˜alpha) and D2 (˜beta) activities of the AD subject compared with the CTL subject are clearly observed through the amplitudes of the corresponding signals.

It is noted that the inventors initially determined EEG features using the traditional short-time FFT with sliding windows of 8-second duration. The inventors then calculated the mean powers, standard deviations, skewness, and kurtosis for all the frequency ranges corresponding to the major brain frequency sub-bands as listed in Table 2. However, the inventors were unable to determine any of the widely reported spectral discriminating features and determined above using DWT except higher mean power.

Among the active states, the discriminating features during auditory stimulation at 18 Hz all belonged to the wavelet coefficient in the D3 scale range. Other discriminating features included skewness of D2 and D3 during the One Card Learning cognitive task (CG3), skewness of D3 during Attention (CG1) task, and kurtosis of D5 during PASAT with 2.0 s interval (P2.0).

Multivariate ANOVA confirmed the null hypothesis for these features but could not reject the hypothesis that these features lie on the same line. In other words, the six dependent variables, features of the wavelet coefficients within the same sub-band, may not be independent discriminants Thus, the wavelet coefficient features within the same sub-bands are highly correlated and the inventors cannot prove that any of the recordings blocks displayed in Table 3 has more than one independent discriminating feature. In general, the low number of independent statistically significant features may be attributed to the small sample size of the study.

In this study a large number of pairwise statistical tests (n=408) have been performed. Hence, the inventors attempted to apply different variations of Bonferroni and False Discovery Rate corrections for multiple comparisons. However, the inventors were unable to determine any significant results for such a large number of tests. Note that, these conservative multiple comparison corrections are not strictly required in exploratory analysis.

### Example 3: Significant Continuous Wavelet Transformation Features

A. Univariate Statistics

Common statistical methods that rely on normal distribution were not applicable in the study. Therefore, the inventors used the nonparametric Wilcoxon rank-sum test, which is the two sample version of the Kruskal-Wallis one-way analysis of variance (ANOVA) by ranks. The null hypothesis of the method is that the populations from which the samples originate have the same median. The test does not identify how many differences actually occur or where they occur.

Initially, the inventors performed univariate analysis (false positive rate p<0.05) on the six sets of features extracted from the seventeen recording sessions based on each of the five different wavelet functions. Since, in each case, a large number of pairwise statistical tests (612) have been performed, multiple comparison adjustment may be applied to reduce the possibility of spurious significant results. Hence, the inventors applied False Discovery Rate (FDR) for multiple comparisons for more rigorous verification of the statistical significant features. Note that these multiple comparison corrections are not strictly required in exploratory analysis and do not prove the significance of the findings. Nonetheless, they minimize the likelihood of the occurrence of false significant findings.

While the sample size in this study is small, N=24, it does not adversely affect the results of the Wilcoxon rank-sum test. Considering the hypothesis in each test that the 10 AD patient features are different from the 14 normal subject features with alpha=0.05 for type I error and 95% test power (type II error beta=0.05) would require a minimum of N=18 subjects. In fact, a sample size of N=24 achieves 98% power with alpha=0.05.

The number of pairwise statistically significant EEG features of AD patients compared to CTL subjects ranged from 63 to 73 depending on the wavelet function. The inventors found very few significant skewness and kurtosis features and very few features for the active state recordings. Hence, the inventors applied FDR to subset of mean power, standard deviation, and entropy features during resting states, which reduced the significant features to the 40 to 50 range. While most features were common, a few differed based on the selected wavelet function. Interestingly, the inventors found very few statistically significant features during EC1, EC7, and EO8 resting states at the very beginning and end of the recording sessions perhaps due to lack of true resting states.

The inventors found very few statistically significant features during the active states. Those of importance were only relative mean powers of the wavelet scales corresponding to theta upper sub-band during CG3 (p=0.040), WE of the scales corresponding to delta_upper sub-band during AS1 (p=0.006), and skewness of scale ranges corresponding to alpha sub-band during AS3. However, none of these findings were found to be significant after FDR adjustments.

In general, the second eye-open resting condition recordings (EO4) yielded the most discriminating features across all wavelet functions with very low false positive rates. A subset of features determined during resting EO2 through EO6 states and active AS1 state are listed in Table 6, derived based on db6 wavelet function, with corresponding false positive rate p-values for the statistically significant features. The features which were found to be statistically significant after FDR adjustment (false positive rate q<0.05) are listed in bold.

Most notably, the results indicated that the relative and absolute mean powers of the wavelet scales corresponding to lower and upper beta sub-band were significantly lower for AD patients when compared to control subjects during resting eyes-open condition. Also, the absolute power of the wavelet scales corresponding the theta sub-band in EO4 and EC5 states were significantly higher for AD patients compared to CTL subjects. These results are consistent with those reported in the literature.

While other studies have found no significant entropy features associated with AD at FP 1 position, interesting new results can be observed regarding WE and SE due to the inventors' classification of these quantities based on scale ranges corresponding to major brain frequency sub-bands. An important result is that WE of scale ranges corresponding to alpha sub-band is significantly lower for AD compared to CTL subjects during EO4 (p=0.001, q=0.027) and EC5 (p=0.0006, q=0.018) recording states where the q-values represent the false positive rate by FDR. The SE of scale ranges corresponding to beta sub-band during EC3 (p=0.016) and theta sub-band during EO4 (p=0.026), EC5 (p=0.047), and EO6 (p=0.035) are all significantly lower for AD compared regardless of the wavelet function. While the SE results could not be verified by FDR, the overall entropy features indicate lower complexity of EEG signals from AD patients when compared to CTL subjects.

B. Comparison with Other Methods

The inventors determined EEG features using the traditional Fast Fourier Transform (FFT) and discrete wavelet transform (DWT). In the case of FFT, the inventors used 8 s (˜1000 sample) sliding Blackman windows and determined the absolute and relative and mean powers, standard deviation, and skewness for the frequency ranges listed in Table 5.

The inventors performed five levels of decomposition for DWT using five mother wavelets from the Daubechies family db2, db4, db6, db8, and db10, which resulted in six sub-bands. The filtered signals in four of these sub-bands approximately represented the EEG major spectral frequency bands, delta-upper, theta, alpha and beta. The inventors then extracted the mean power, standard deviation, and skewness of the wavelet coefficients as the features.

A shorter list of the common statistical features between the three approaches, the absolute mean powers of major brain sub-bands, are listed in Table 7 where only db6 wavelet results are shown for CWT and DWT approaches. It is clear that FFT is not able to capture most of the statistically significant features identified by CWT and DWT. In fact, after multiple comparison adjustments by FDR, no reliable discriminant feature could be reported. This clearly indicated that wavelet transform has a superior performance in classification of AD patients when compared to FFT.

DWT feature are, however, comparable with CWT where both determine similar discriminating features. While DWT seem to identify absolute mean power as a discriminating feature, the result could not be confirmed by FDR. Another disadvantage of DWT is that it could not be used to determine features corresponding to more detailed upper and lower sub-bands.

C. Multivariate Statistics

The inventors used multivariate ANOVA to investigate the correlation between the statistically significant features from the univariate analysis. The inventors grouped the five features (absolute and relative mean powers, standard deviation, skewness) corresponding to each CWT scale range listed in Table 5 as the five variables of multivariate analysis. In addition, the inventors grouped wavelet and sample entropy corresponding to each CWT scale range as the two variables for separate multivariate analysis. In both cases, the multivariate analysis consistently confirmed univariate results. However, multivariate ANOVA could not reject the hypothesis that the variable in each group lie on the same line. In other words, the five dependent variables, absolute and relative mean powers, standard deviation, and skewness features of the wavelet coefficients within the same sub-band, may not be independent discriminants. Similarly the wavelet and sample entropy features of the wavelet coefficients within the same sub-band, may not be independent discriminants.

D. Decision Tree

Since numerous statistically significant discriminating features were identified in the study, the inventors used the decision tree algorithms to determine the most dominant and reliable ones. Decision tree analysis holds several advantages over traditional supervised methods, such as maximum likelihood classification. Decision tree is a non-parametric method in that it does not depend on assumption of data distribution. Another advantage is its ability to handle missing values, which is a very common problem in dealing with the biomedical data. The most important component of a decision tree induction strategy is the split criterion, which selects an attribute test that determines the distribution of training objects into sub-sets consequently leading to sub-trees.

In this study, the inventors used three well-known split criteria: Gini, Twoing, and maximum deviance reduction (or entropy) indexes. The inventors applied the three algorithms to each set of 612 CWT features derived based on the five different mother wavelets.

In other words, a decision tree was derived through comparison of 6120 AD samples (612 features for 10 subjects) with 8568 CTL samples (612 features for 14 subjects) for each mother wavelet and each decision tree algorithm for a total of fifteen trees.

The top line result of the decision tree algorithm for comparing the AD and CTL subjects is shown in FIG. 2 with the number of classified subjects indicated within parentheses. The result indicates that absolute mean power of the wavelet scales corresponding to 4-8 Hz (theta sub-band) of the second eyes open state (EO4) is the most dominant discriminating feature of AD patients. The tree implies that if the absolute power of CWT coefficients of the scale range corresponding to theta sub band during EO4 of a subject is greater than 3.71, in arbitrary units (arb), then the subject is identified to have AD. The result is consistent across all five wavelet functions and all three split criteria. The reliability of this classification is further reinforced since the feature was determined to be statistically significant (p=4e-5, q=0.001) and other studies have also found the absolute theta band mean power to be significantly higher for AD patients when compared to control subjects.

In order to determine the next dominant feature, the inventors removed the most dominant discriminating feature and re-applied the decision tree algorithms. The new decision tree is shown in FIG. 3, which implies that if the standard deviation of CWT coefficients corresponding to theta sub-band during EO4 of a subject is greater than 1.91 arb, then the subject has AD. This feature was also determined to be statistically significant (p=4e-5, q=0.001) and the decision tree result was consistent across all five wavelet functions and the three splitting criteria.

E. Comparison with DWT

The inventors also applied the three decision tree algorithms to features extracted through DWT decomposition with db2 through db10 wavelet functions using the same three split criteria. Surprisingly, the top line decision tree uses a combination of three features to classify AD patients which included two statistically insignificant features. When the inventors excluded the active state recordings, the top line result was the same as the one shown in FIG. 2. However, three subjects were misclassified. This clearly indicated that CWT is much more suitable for classification of AD patients compared to DWT in the pilot study.

F. A New Classification Index

Since multivariate ANOVA did not establish the independence of the standard deviations and mean powers within the same scale ranges, the inventors removed the first and second most dominant discriminating features to identify additional independent significant features. In this case, however, the decision tree results were not as straightforward and depended on the selected wavelet function and split criterion. In some cases, the features were not statistically significant while other cases involved false classifications. Thus, the inventors defined an index, I_{cn}, that penalized the decision tree for having too many features since the probability of false positives increases as the number of features increases:

$${I}_{cn} = \{ \begin{matrix}1 , & {n}_{f} = 1 \\ 2 , & {n}_{f} = 2 \\ 3 , & {n}_{f} = 3 \\ 4 , & {n}_{f} \geq 4\end{matrix}$$

where n_{f} is the number of selected features. The inventors defined a second index, I_{cp}, to penalize the decision tree based on the number of incorrectly classified subjects as a fraction of total number of subjects in that group:

$${I}_{cp} = \{ \begin{matrix}0 , & {n}_{i} = 0 \\ 1 , & 0 < {n}_{i} \leq 0.1 \\ 2 , & 0.1 < {n}_{i} \leq 0.2 \\ ⋮ & ⋮ \\ 6 , & {n}_{i} > 0.5\end{matrix}$$

where ni represents the fraction. Finally, the inventors considered the statistical significance of the discriminating features used in a decision tree as the most important factor in the classification reliability. Hence, the inventors penalized the decision tree, with index Ics:

$${I}_{cs} = \{ \begin{matrix}0 , & {n}_{s} = 0 \\ 2 , & 0 < {n}_{s} \leq 0.25 \\ 4 , & 0.25 < {n}_{s} \leq 0.4 \\ 6 , & 0.4 < {n}_{s} \leq 0.5 \\ 8 , & {n}_{s} > 0.5\end{matrix}$$

where ns is the fraction of number of statistically insignificant features over total number of features in the decision tree. The new index was computed as sum of the indexes defined for the above three categories as: Ic=Icn+Icp+Ics (Eq. (7)). Hence, the minimum value of the index is 1 which is the case for the decision trees introduced in FIGS. 2 and 3.

The inventors applied the new index to select the most reliable decision tree based on the choice of wavelet function and split criterion. Table 8 shows the index values for all 15 cases, which indicate that any of the three split criteria and wavelet functions db4, db6, and db8 provide the most reliable decision tree shown in FIG. 4. The tree indicates that if the wavelet entropy of CWT coefficients of corresponding to 8-13 Hz (˜alpha sub-band) during EO4 for a subject is less than 1.6 arb, then the subject has AD. This feature was also determined to be statistically significant (p=0.0006, q=0.027), as listed in Table 6. However, one CTL subject was incorrectly classified as an AD patient resulting in an index value of 2.

TABLE 8
Index values after removal of the first two dominant features

| — | Gini | Twoing | Deviance |
|---|---|---|---|
| Morlet | 8 | 8 | 8 |
| Db4 | 2 | 2 | 2 |
| Db6 | 2 | 2 | 2 |
| Db8 | 2 | 2 | 2 |
| Db10 | 12 | 8 | 12 |

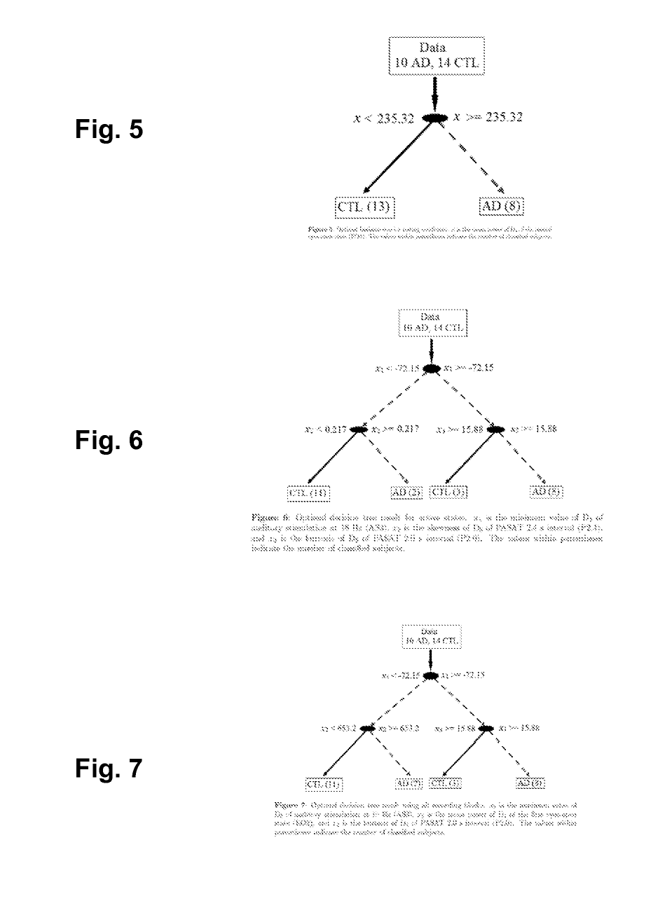

Next, the inventors removed the first three most dominant discriminating features to identify more significant features. Table 9 shows the classification indexes across the five wavelet functions and the three split criteria where db6 wavelet function provides the best classification regardless of split criterion. The resulting decision tree for this fourth level of classification, shown in FIG. 5, implies that if wavelet entropy of CWT coefficients corresponding to 2-4 Hz (delta upper sub-band) during AS1 recording of a subject is less than 2.63 arb, then the subject is identified as an AD patient. Otherwise, if the skewness value of the wavelet coefficients corresponding to 2-4 Hz from the EO4 is less than −0.022 arb, then the subject is again identified as an AD patient (the dashed lines in decision tree).

TABLE 9
Index values after removal of the first three dominant features

| — | Gini | Twoing | Deviance |
|---|---|---|---|
| Morlet | 8 | 8 | 8 |
| Db4 | 6 | 10 | 6 |
| Db6 | 5 | 5 | 5 |
| Db8 | 14 | 14 | 14 |
| Db10 | 14 | 14 | 14 |

The second feature, skewness of CWT coefficients in the delta-upper sub-band during EO4 was determined to be statistically significant (p=0.035, q=0.046). While the first feature, WE of CWT coefficients in the delta-upper sub-band during AS1, was only determined to be statistically significant through univariate ANOVA (p=0.006) but could not be confirmed when adjusted by FDR. Also, two CTL subjects where incorrectly classified among the 7 AD patients of the first split and two features were employed resulting in index value of 5. Hence, the search for further features was not useful since the resulting optimal decision trees used one or more statistically insignificant features along with several incorrect classifications.

G. Internal cross validation

The inventors randomly left one test subject out (e.g., control subject 5) and re-applied the decision tree algorithms to all features of the remaining subjects as the training set. The algorithm derived the same decision trees at all four levels presented in FIGS. 2-5. There were no false classifications when the inventors applied the first three decision trees to the randomly selected control subject. In the fourth case (FIG. 5), however, false classification is possible depending which subject is left out.

Those skilled in the art will appreciate that the invention may be applied to other applications and may be modified without departing from the scope of the invention. Accordingly, the scope of the invention is not intended to be limited to the exemplary embodiments described above, but only by the appended claims.

What is claimed:

1. A method of extracting brain frequency sub bands corresponding to medical condition from EEG time series data of a patient, comprising:
applying wavelet transforms to the EEG time series data to generate a continuous wavelet transformation (CWT) time series at each wavelet scale;
calculating Wavelet Entropy (WE) and Sample Entropy (SE) directly from the CWT time series at each wavelet scale;
calculating arithmetic or geometric means and accumulations across scale ranges of interest; and
selecting data from major brain frequency sub-bands as candidate sets of extraction features for analysis as a diagnostic signature for the medical condition.

2. The method of claim 1, wherein WE is calculated for each of delta upper, theta, alpha, and beta sub-bands and used as a candidate set of extracted features.

3. The method of claim 1, SE is calculated when applied to a time series representing wavelet coefficients at each wavelet scale after CWT rather than to the raw EEG voltage as a function of time.

4. The method of claim 1, further comprising removing areas of artifact from an EEG time series by nullifying an artifact region and then reconstructing the nulled samples using FFT interpolation of trailing and subsequent recorded EEG time series data.

5. The method of claim 1, wherein the candidate sets of extraction features for analysis as a diagnostic signature for Alzheimer's disease comprise a wavelet coefficient in a D3 scale range during a binaural beat auditory stimulation at beat frequency of 18 Hz;
skewness of D2 and/or D3 scale during a One Card Learning cognitive task (CG3), skewness of D3 during a CogState Attention (CG1) task, or a kurtosis of a D5 scale during a PASAT task.

6. The method of claim 1, wherein the candidate sets of extraction features for analysis as a diagnostic signature for Alzheimer's disease comprise relative mean powers of the wavelet scales corresponding to theta_upper sub-band during CG3 (p=0.040), the WE of the wavelet scales corresponding to delta upper sub-band during AS 1 (p=0.006), and skewness of wavelet scale ranges corresponding to alpha sub-band during AS3 (p=0.034).

7. The method of claim 1, wherein the diagnostic signature for Alzheimer's disease (AD) comprises WE of CWT scale ranges corresponding to an alpha sub-band that is significantly lower for AD compared to CTL subjects during an Eyes Open task and/or an Eyes Closed task.

8. The method of claim 1, wherein the diagnostic signature for Alzheimer's disease (AD) comprises SE of CWT scale ranges corresponding to a beta sub-band during an Eyes Closed task (EC3) and theta sub-band during an Eyes Open task (EO4, EO6) or Eyes Closed task (EC5) that are significantly lower for AD regardless of the wavelet function compared to CTL subjects.

9. The method of claim 1, wherein the diagnostic signature for Alzheimer's disease (AD) comprises a standard deviation of CWT coefficients corresponding to a theta sub-band during an Eyes Open task (EO4) and when the standard deviation is greater than 1.91 arb, then the subject is predicted to have AD.

10. The method of claim 1, wherein the diagnostic signature for Alzheimer's disease (AD) comprises WE of CWT coefficients corresponding to 8-13 Hz during an Eyes Open task and, if a value of WE is less than 1.6 arb, then the subject is predicted to have AD.

11. The method of claim 1, wherein the diagnostic signature for Alzheimer's disease (AD) comprises WE of CWT coefficients corresponding to 2-4 Hz during a binaural beat auditory stimulation task (AS1) and if the value of WE for a subject is less than 2.63 arb, then the subject is predicted to have AD.

12. The method of claim 1, wherein the diagnostic signature for Alzheimer's disease (AD) comprises a skewness value of CWT coefficients corresponding to 2-4 Hz from an Eyes Open task and if the skewness value is less than −0.022 arb, then the subject is predicted to have AD.
